(19) United States
(12) Patent Application Publication
(10) Pub. No.: US 2011/0125263 A1
(43) Pub. Date: May 26, 2011

(54) METHOD FOR PRODUCING NANOSTRUCTURES ON A SURFACE OF A MEDICAL IMPLANT

(75) Inventors: Thomas J. Webster, Barrington, RI (US); Chang Yao, Jiangsu (CN)

(73) Assignee: BROWN UNIVERSITY, Providence, RI (US)

(21) Appl. No.: 12/674,852

(22) PCT Filed: Aug. 22, 2008
(86) PCT No.: PCT/US08/73963

§ 371 Date: Feb. 8, 2011

Related U.S. Application Data
(60) Provisional application No. 60/957,726, filed on Aug. 24, 2007.

(51) Int. Cl. A61F 2/02; C25D 5/34

(52) U.S. Cl. 623/11.11; 205/205

(57) ABSTRACT

A method for treating a surface of a medical implant to create nanostructures on the surface that results in increased in-vivo chondrocyte adhesion to the surface. Further, disclosed is a method to fabricate a drug delivery system. The drug delivery system includes a medical implant that has undergone a surface treatment process that results in the modification of the surface configuration and topography. The modified surface acts as a depot or reservoir for loaded biological material, biologic agents or pharmaceutical products. Additionally, a device for delivering pharmaceutical products or other biological materials is disclosed. The device includes integrally attached nanostructures that retain or adsorb the loaded pharmaceutical products and/or biological materials. Further disclosed is a medical implant that includes a surface configured to allow for and regulate protein adsorption. The surface of the medical implant has a layer of nanostructures rigidly attached with varying porosity and orientation that allow for surface protein adsorption to be controlled.

### TECHNICAL FIELD

This invention relates, in general, to modifying a surface of a substrate material, and in particular, to an anodization method for treating the surface of an implantable device to increase in-vivo functionality, including chondrocyte adhesion, protein adsorption and drug delivery.

### BACKGROUND OF THE INVENTION

Certain materials can be improved for use in medical applications. For example, resulting changes in topography to a titanium substrate from oxidation can increase biologically-inspired nanometer surface roughness for better protein adsorption, osteoblast attachment with eventual osseointegration and chondrocyte adhesion. Further, the use of medical implants as drug delivery mechanisms is an attractive alternative to current methodologies.

It is well known that titanium is known as a “valve metal”, i.e. when it is exposed to air, water and other oxygen containing atmospheres, an oxide layer spontaneously forms on its surface to protect the underlying metal. For this reason, titanium-based alloys have excellent corrosion resistance and good biocompatibility. Also, due to its light weight and appropriate mechanical properties, titanium and its alloys are widely used in orthopedic applications. It would be advantageous to use the same titanium to regenerate bone and cartilage as the use of one material to regenerate bone and another material to regenerate cartilage within the same device may necessitate the use of a coating which can delaminate during articulation. In addition, titanium has good wear properties and when oxidized could interact well with lubrican (a lubricating hydrophilic protein found in articulating joints). However, the inability of chondrocytes (cartilage synthesizing cells) to adhere and subsequently form new cartilage tissue on titanium has remained problematic. Clearly, for such patients who simultaneously have bone and cartilage tissue damage, a titanium-based implant that can serve to regenerate both tissues would be most beneficial.

It is well understood that interactions between implants and cells, specifically osteoblasts mainly depend on surface properties like topography, roughness, chemistry, and wettability. To improve implant integration into surrounding bone and cartilage, various surface treatments have been attempted with limited success to modify the topography and chemistry of titanium. Other studies have also focused on the geometry of the anodized structures formed on titanium.

Cartilage tissue possesses a unique nanostructure rarely duplicated in synthetic materials. Specifically, chondrocytes are naturally accustomed to interacting with a well-organized nanostructured collagen matrix. Despite the role that titanium currently plays in both orthopedic and cartilage applications, and the natural nanostructure of cartilage, no reports exist investigating chondrocyte functions on titanium anodized to possess biologically-inspired nanotubes.

Developing a novel method of enhancing in-vivo functionality for various materials, specifically to improve a material's chondrocyte adhesion properties, increase a material's ability to regulate protein adsorption on a surface and also to allow a material to function as a drug delivery mechanism would be desirable.

### SUMMARY OF THE INVENTION

The present invention provides in one aspect, a method for producing a plurality of nanostructures on a surface of a medical implant. The method includes the step of presoaking the implant in a solution. The method includes the further steps of providing an anodization electrolyte solution and a cathode. The method also includes the steps of submerging the cathode and medical implant in the electrolyte solution and then applying a voltage for a set time period between the medical implant and the cathode to generate a plurality of nanostructures on the surface of the medical implant. Further, the method includes the step of removing the medical implant from the electrolyte solution and rinsing the surface of the medical implant.

The present invention provides in another aspect, a method for fabricating a medical implant with enhanced or increased in vivo chondrocyte functionality. The method includes the step of obtaining a medical implant with the medical implant being fabricated from a metallic material, a polymer, a ceramic or a composite. The method also includes the step of treating the surface of the medical implant to modify the surface configuration, roughness or topography that then results in increased chondrocyte adhesion.

The present invention provides in yet another aspect, a method for fabricating a drug delivery system. The method may include the step of obtaining a medical implant, with the medical implant being made from either a metallic material, preferably titanium or a titanium alloy, a polymer, a ceramic or a composite. The method may also include the step of treating a surface of the medical implant to modify the surface configuration or topography resulting in increased surface roughness. Such surface modification results in the fabrication of a system that delivers biological materials and/or pharmaceutical products within the body.

Yet another aspect of the present invention provides, a device for delivering a pharmaceutical product or biologic agent within a living being that includes a medical implant having a surface to which is attached a multitude of nano structures. The nanostructures are arranged in a manner to retain and/or adsorb the pharmaceutical product or biologic agent that has been loaded onto/into the nanostructure by a separate process.

Yet a further aspect of the present invention includes, a medical implant that has a surface configured for allowing for and regulating protein adsorption. The surface may include a multitude of nanostructures with these nanostructures being formed and fixed to the surface after the implant has undergone a surface anodization treatment process.

These and additional features and advantages are realized through techniques and use of the present invention. Other embodiments and aspects of the present invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a schematic showing the anodization process and vessel in which the two electrode configurations are linked to a DC power supply. A platinum mesh and titanium disk served as the cathode and anode, respectively with 1.5% HF used as an electrolyte contained in a Teflon beaker, in accordance with an aspect of the invention;

FIGS. 2(a), (b) and (c) are scanning electron microscopy images of: (a) un-anodized titanium; (b) nanotubular anodized titanium (low magnification); and (c) nanotubular anodized titanium (high magnification). Bars=1 μm for un-anodized Ti, and 200 nm (low magnification) and 500 nm (high magnification) for nanotubular anodized titanium, in accordance with an aspect of the invention;

FIGS. 3(a) and (b) are AFM images of: (a) un-anodized titanium; and (b) anodized titanium with nanotube-like structures. The scan area is 1×1 μm, in accordance with an aspect of the invention;

FIG. 4. is a bar graph showing increased chondrocyte adhesion on nanotubular anodized titanium. Values are mean±SEM; n=3; * p<0.01 compared to the glass (reference); ** p<0.01 compared to un-anodized titanium, in accordance with an aspect of the invention;

FIGS. 5(a) and (b) are bar graphs: (a) shows fibronectin; and (b) vitronectin, respectively adsorption on un-anodized titanium, anodized titanium possessing nano-particulate structures (0.5% HF, 10 V and 20 min), and anodized titanium possessing nano-tubular structures (0.5% HF, 20 V and 20 min). Values are mean±SEM; n=3; *p<0.1 (compared to un-anodized titanium) and #p<0.1 (compared to nano-particulate structures); in accordance with an aspect of the invention;

FIG. 6 is a schematic showing the silanization process for anodized titanium, in accordance with an aspect of the invention;

FIGS. 7(a), (b), (c), and (d) are images of SEM micrographs that reveal unchanged nanotubular structures after three steps of chemical modifications: (a) Original anodized titanium in 1.5% HF for 10 minutes; (b) anodized titanium that underwent hydroxylation in a Piranha solution for 5 minutes; (c) the sample in (b) that has undergone silanization; and (d) the surface of sample (c) that has undergone the replacement of amine groups with methyl groups. Scale bars=200 nm., in accordance with an aspect of the invention;

FIG. 8 shows the CBQCA reagent that has confirmed the amine termination after silanization of the anodized titanium, in accordance with an aspect of the invention;

FIGS. 9 are images of SEM micrographs that show the filled/unfilled nanotubes after being loaded with penicillin drug molecules on the A, A-OH, A-NH, and A-CH₃ substrates, in accordance with an aspect of the invention;

FIGS. 10(a), (b), (c), (d) and (e) show images of SEM micrographs of the partially abraded titania nanotubular structures: (a) anodized titanium possessing nanotubular structures; (b) anodized titanium loaded with P/S showed some unfilled nanotubes in the middle portion; (c) A-OH loaded with P/S showed filled nanotubes; (d) A-NH₂ loaded with P/S showed some unfilled nanotubes on the top and in the middle portion; and (e) A-CH₃ loaded with P/S showed some unfilled nanotubes on the top and in the middle portion, in accordance with an aspect of the invention;

FIGS. 11(a) and (b) show two bar graphs indicating the release of: (a) P/S and (b) P-G from the five various titanium substrates after 1 hour, 2 hours, 1 day, and 2 days using the physical adsorption method. #p<0.1 compared to un-anodized titanium, ##p<0.1 compared to anodized titanium with nanotubular structures, *p<0.1 compared to respective release amount after 2 hours, **p<0.1 compared to respective release amount after 1 day, ***p<0.1 compared to respective release amount after 2 days. Data=Mean+SEM, N=3, in accordance with an aspect of the invention;

FIGS. 12(a), (b), (c), (d) and (e) show images of SEM micrographs of: (a) anodized titanium substrates soaked in a 5% P/S solution for 30 minutes; (b) anodized titanium electrodeposited in a 0.9% NaCl solution for 5 minutes under 8 V; (c) anodized titanium electrodeposited in a 5% P/S solution for 5 minutes under 8 V; (d) anodized titanium terminated with —OH electrodeposited in a 5% P/S solution for 5 minutes under 8 V; (e) anodized titanium terminated with —NH₂ electrodeposited in a 5% P/S solution for 5 minutes under 8 V; and (f) anodized titanium terminated with —CH₃ electrodeposited in a 5% P/S solution for 5 minutes under 8 V, in accordance with an aspect of the invention;

FIGS. 13(a) and (b) show two bar graphs indicating the release of: (a) P/S; and (b) P-G from the five various titanium substrates after 1 hour, 2 hours, 1 day, and 2 days using the electrodeposition method. Data=Mean+SEM, N=3. *p<0.1 compared to respective release amount after 2 hours, in accordance with an aspect of the invention;

FIGS. 14 is a schematic of the steps to co-precipitate antibiotics with apatite crystals in a 1.5×SBF solution (co-precipitation drug loading method), in accordance with an aspect of the invention;

FIGS. 15(a), (b), (c), (d), (e) and (f) show images of SEM micrographs of: (a) anodized titanium; (b) anodized titanium soaked in 6M NaOH for 1 hour; (c) and (d) ASH samples soaked in 1.5×SBF for 3 days without P/Sand; (e) and (f) ASH sample soaked in 1.5×SBF for 3 days with 20% P/S. ASH=anodized, soaked in NaOH and heat treated titanium samples, in accordance with an aspect of the invention;

FIGS. 16 shows an EDS spectrum of the ASH titanium samples that reveal the existence of Ca and P in the coatings deposited onto the anodized titanium surfaces during the co-precipitation drug loading method. ASH=anodized, soaked in NaOH and heat treated titanium samples, in accordance with an aspect of the invention;

FIGS. 17(a), (b), (c) and (d) show images of SEM micrographs of anodized titanium surfaces co-precipitated with P/S and minerals, specifically: (a) the nanotube structures following abrasion to show the cross-section and the middle portion of the titania nanotubes were not filled with drugs or minerals after the co-precipitation process; (b) to (d) are top views of the anodized titanium samples following co-precipitated with 5%, 10%, and 20% P/S in the SBF solution after 21 days of release, in accordance with an aspect of the invention; and

FIG. 18 shows a bar graph of the results following the measurement of the released penicillin amounts after different time periods from anodized titanium co-precipitated with 5%, 10%, and 20% penicillin/SBF solution; #p<0.1 compared to 5 and 10% data after 1 hour; ##p<0.1 compared to 2 hours, 1 day, 5 days, 7 days, 15 days, and 21 days of 20% data series; *p<0.1 compared to 2 hours, 1 day, 15 days, and 21 days of 5% data series; **p<0.1 compared to 2 hour, 1 day, 15 days, and 21 days of 10% data series; ***p<0.1 compared to 2 hours, 15 days, and 21 days of 10% data series. Data=Mean+SEM, N=3, in accordance with an aspect of the invention.

### DETAILED DESCRIPTION OF THE INVENTION

The following description is intended to convey an understanding of the various embodiments of the invention by providing several examples and details of the nanoscale surface that results from the inventive methodology.

The present invention provides a method for treating a surface of an implant to modify the surface characteristics by forming titanium nanotubes following the material undergoing an anodization procedure. The unique surface characteristics of the formed oxide nanotubes resulting in many structural advantages for the user of the treated medical implant.

The present invention is also based in part on the surprising discovery that medical implants that include a surface composed of anodized nanotubular titanium have been shown to have increased cellular activity around that medical implant following implantation. It should be noted that it would be well understood by one skilled in the art that other substrate materials may be used and undergo the subject method for surface topography change and resultant cellular enhancement, with these materials including, but are not being limited to other titanium alloys, cobalt chromium alloys, stainless steel alloys, composites, and polymers.

The present invention also would include a medical implant on which such process was performed, thus enhancing the cytocompatibility of the medical implant post-implantation.

Also, as disclosed herein, the present invention is also based in part on the unexpected result that the changed topography of the implant surface creates a unique drug delivery mechanism on said surface of the medical implant, wherein the formed nanotubes function as drug reservoirs, whereby modifying the size, depth and density of the nanotubes will allow for customization for the rate of release of embedded drugs. The treated medical implant thus acting as an innovative drug delivery system for the patient. The present invention yet further provides for a medical implant that results from the performance of the disclosed anodization method to regulate protein adsorption and resulting cellular interaction on the surface of the device following implantation.

The features and other details of the various embodiments of the invention will now be more particularly described with references to the accompanying drawings, experimentation results, examples and claims. Certain terms are defined throughout the specification. Unless otherwise defined, all technical and scientific terms used herein have the same meaning as commonly understood by one of ordinary skill in the art to which this invention pertains. In some cases, terms with commonly understood meanings are defined herein for clarity and/or for ready reference, and the inclusion of such definitions herein should not necessarily be construed to represent a substantial difference over the definition of the term as generally understood in the art. Furthermore, as used herein and in the appended claims, the singular forms include plural referents unless the context clearly dictates otherwise. Thus, for example, reference to “titanium nanotube” includes one or more of such titanium nanotubes, as would be known to those skilled in the art.

Discussed below is the novel evaluation undertaken by the inventors that more fully describes the present invention of an anodization method for treating a surface of a titanium medical implant that causes a changed topography and results in enhanced or increased chondrocyte adhesion, as well as another aspect of the invention, a medical implant that has undergone the anodization process resulting in the implant surface being capable to regulate protein adsorption. A further aspect of the invention is a medical implant that again has undergone the inventive process, the resulting implant surface being a new and novel drug delivery mechanism.

### Materials and Methods

1. Titanium substrates

Titanium foil (10×10×0.2 cm; 99.2% pure; Alfa Aesar) was cut into 1×1 cm squares using a metal abrasive cutter (Buchler 10-1000; Buehler LTS, IL). All the substrates were then cleaned with liquid soap (VWR) and 70% ethanol (AAPER) for 10 minutes in an aqua sonicator (Model 50 T; VWR). Substrates were then dried in an oven (VWR) at about 65° C. for 30 minutes to prepare them for anodization. After anodization, all the substrates were ultrasonically washed in an aqua sonicator with acetone (Mallinckrodt) for 20 minutes and 70% ethanol for 20 minutes.

Borosilicate glass (Fisher Scientific; 1.8 cm diameter) was used as a reference material in the present study. The glass coverslips were degreased by soaking in acetone for 10 minutes, sonicating in acetone for 10 minutes, soaking in 70% ethanol for 10 minutes, and sonicating in ethanol for 10 minutes. Lastly, the coverslips were etched in 1 N NaOH (Sigma) for 1 hour at room temperature.

### 2. Anodization Process

In order to create the nanotubes, prior to anodization, the titanium substrates were immersed in an acid mixture (2 ml 48% HF, 3 ml 70% HNO₃ (both Mallinckrodt Chemicals) and 100 ml DI water) for 5 minutes to remove the naturally formed oxide layer. Some of the acid-polished substrates were then immediately treated by anodization.

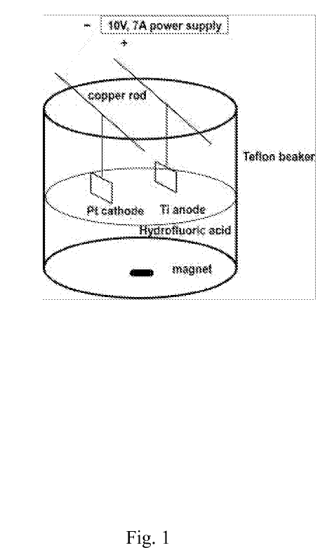

As shown in FIG. 1, the titanium substrates served as an anode in the anodization process while an inert platinum sheet (Alfa Aesar) was used as a cathode. The anode and cathode were connected by copper wires and were linked to a positive and negative port of a 30V/3 A power supply (SP-2711; Schlumberger), respectively. During processing, the anode and cathode were kept parallel with a separation distance of about 1 cm, and were submerged into an electrolyte solution in a Teflon beaker (VWR). Dilute hydrofluoric acid (1.5 wt %) was used as an electrolyte.

It is understood by one skilled in the art that the resulting anodized titanium structures are determined by the values of various parameters and that it is necessary to keep certain process variables constant in order to form titanium nanotubes. For example, the potential between the anode and cathode was kept constant at 20 volts. All anodizations were completed for 20 minutes for this particular evaluation. After anodization was completed, all substrates were rinsed thoroughly with deionized (DI) H₂O, dried in an oven at about 65° C. for 30 minutes, and sterilized in an autoclave at 120° C. for 30 minutes.

An alternative embodiment of the process invention for producing an implant with titanium nanotubes may include the following step parameters: obtaining a substrate surface having a planar configuration or being three-dimensional (i.e., possesses an inner surface or layer) in orientation and construction; pre-treating the substrate by soaking the substrate in 1% HF and 2% HNO₃ in DI water; using an anodization electrolyte solution: Hydrofluoric acid (0.5%-2%); applying a voltage of 10-25 V for a time of 5 to 30 minutes; rinsing the substrate with acetone and ethanol; keeping the temperature during anodization process at or about room temperature; and using a platinum cathode and Titanium (or its alloys) as the anode. Typically, during the anodization process the voltage is kept constant and the current is allowed to vary. Depending upon the thickness of the oxide layer, the current may vary between 0.05 and 0.15 A for a 1 square cm sample size.

### 3. Substrate Surface Characterization

Surface morphologies of the un-anodized and anodized titanium substrates were mainly characterized using a JEOL JSM-840 Scanning Electron Microscope and a Hitachi S4800 Field Emission Scanning Electron Microscope for ultra-high magnifications. All samples were sputter-coated with AuPd before imaging using a HUMMER I sputter-coater for 3 minutes.

Surface roughness of the titanium substrates was measured by an Atomic Force Microscope (AFM, Multimode SPM Digital Instruments Veeco). The typical tip (NSC15; Mikromasch) curvature radius used in the present study was less than 10 nm. The measurements were conducted in ambient air under tapping mode with a scan rate of 2 Hz. The scan area was 1×1 μm. The root mean square (rms) roughness, relative surface area, and z direction depth were estimated with the aid of Nanoscope imaging software.

To determine the composition of surface oxide formed on titanium, both un-anodized and anodized nanotubular substrates were also examined by an X-ray Photoelectron Spectroscope (XPS, Surface Science Instruments X-probe Spectrometer). This instrument has a monochromatized Al Kα X-ray and a low energy electron flood gun for charge neutralization. X-ray spot size for these acquisitions was on the order of 800 μm. The take-off angle was ˜55°; a 55° take-off angle measures about 50 Å sampling depth. The Service Physics ESCAVB Graphics Viewer program was used to determine peak areas.

Phase analysis of the titanium substrates was carried out by X-ray diffraction (XRD) analysis using a Siemens D500 Diffractometer (Bruker AXS Inc., WI). Copper Kα radiation (λ=1.5418 Å) scanned the nanotubular anodized samples from 2θ angles of 20° to 60° at a scan speed of 0.5°/min with a 0.05° increment. Resulting XRD spectra were compared to titanium (JCPS # 050682) and titania (rutile and anatase; JCPS # 211276 and JCPS # 211272, respectively) standards.

### 4. Cell Experiments

Human articular chondrocytes (cartilage-synthesizing cells; Cell Applications Inc.) were cultured in Chondrocyte Growth Medium (Cell Applications Inc.). Cells were incubated under standard cell culture conditions, specifically, a sterile, humidified, 5% CO₂, 95% air, 37° C. environment. Chondrocytes used for the following experiments were at passage numbers below 10. The phenotype of these chondrocytes has previously been characterized by the synthesis of Chondrocyte Expressed Protein-68 (CEP-68) for up to 21 days in culture under the same conditions. Chondrocytes were seeded at 3,500 cells/cm² pre samples and were allowed to attach for 4 hours. After the prescribed time point, non-adherent cells were removed by rinsing with a phosphate buffered saline (PBS) solution. Cells were then fixed, stained with rhodamine phalloidin, and counted according to standard procedures. Five random fields were counted per substrate and all experiments were run in triplicate, repeated at least three times.

### Results

### 1. Creation of Anodized Titanium Surfaces Possessing Nanotubular Structures

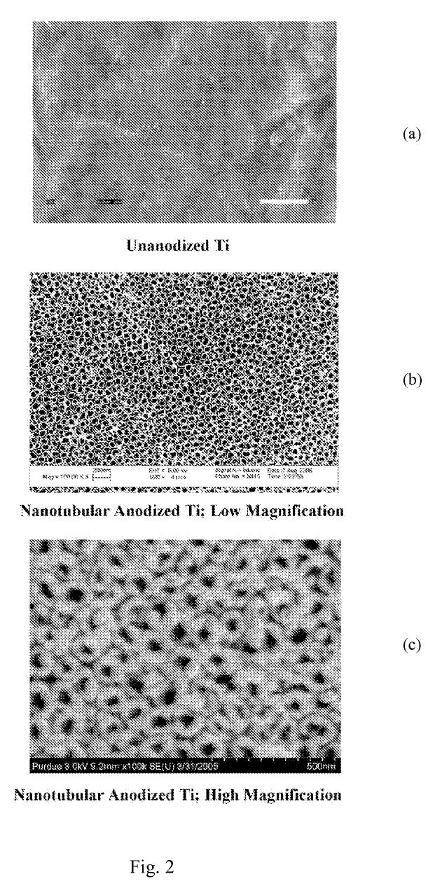

As seen in FIG. 2(a), the un-anodized titanium as purchased from the vendor possessed micron rough surface features as displayed under SEM. After anodization in 0.5% HF at 20 V for 20 minutes, the titanium surface was oxidized and possessed nanotubular structures uniformly distributed over the whole surface (See, FIG. 2(b)). As estimated from these SEM images, FIG. 2(c) shows the inner diameter of the nanotubular structures being from 70 to 80 nm.

### 2. Surface Characterization of Anodized Titanium Substrates

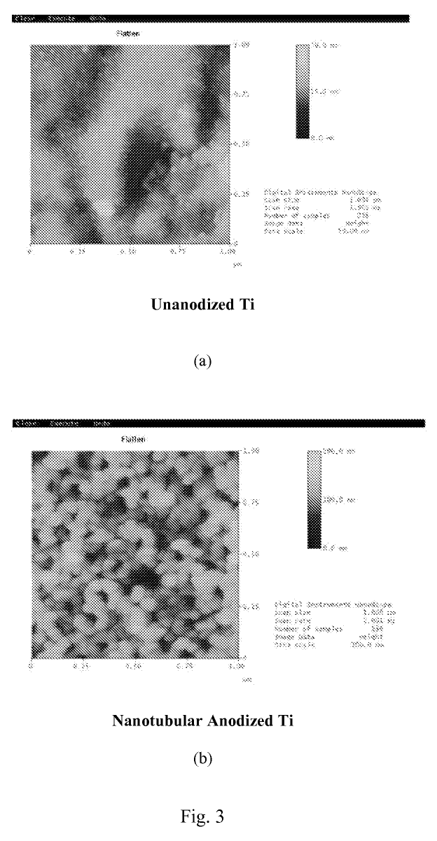

As seen in FIGS. 3(a) and 3(b) and listed on Table 1 below, representative AFM images of un-anodized and nanotubular anodized titanium were characterized by root mean square (rms) and relative surface area. Results showed that the un-anodized titanium surface was relatively smooth (4.74 nm) compared to the nanotubular anodized titanium surfaces. Moreover, the rms value was larger for the nanotubular anodized titanium surface structures (25.54 nm). Further information on the depth and diameter of the nanometer surface features was obtained from the AFM images and profiles. It was estimated that the nanotubes were between 100 and 200 nm deep and had an inner diameter approximately 70 to 80 nm, as also confirmed by SEM.

TABLE 1
Surface roughness of un-anodized and nanotubular
anodized titanium surfaces

| | Relative | Root mean square |
|---|---|---|
| Substrates | surface area | roughness (nm) |
| Un-anodized titanium | 1.018 ± 0.008 | 4.74 ± 1.87 |
| Anodized titanium with | 1.811 ± 0.133* | 25.54 ± 3.02* |
| nano-tube structures | | |

*p < 0.01 compared to un-anodized titanium.

High resolution X-ray Photoelectron Spectroscopy spots were taken on each sample to examine Ti 2p binding energy (See, Table 2 below). Importantly, other than TiO₂, no other titanium species (for example, TiO and Ti₂O₃) were present. X-ray Photoelectron Spectroscopy results also demonstrated that the outermost layers of oxide mainly contained C, O, Ti, F, and N (See, Table 3 below) and were similar between the un-anodized and nanotubular anodized titanium. XRD spectra confirmed the presence of amorphous titania (no anatase or rutile phase was observed) on both un-anodized and nanotubular anodized titanium (data not shown). In summary, it is seen that while the degree of nanometer roughness was much greater for nanotubular anodized titanium compared to un-anodized, chemistry and crystallinity were similar.

TABLE 2
Binding energy of the high resolution Ti 2p peaks for
un-anodized and nanotubular anodized titanium substrates as
examined by X-ray Photoelectron Spectroscopy

| | | Binding | |
|---|---|---|---|
| | | Energy | Area |
| Substrates | Peak | (ev) | % |
| Un-anodized titanium | Ti 2p3/2 | 458.8 | 67.8 |
| | Ti 2p1/2 | 464.5 | 32.1 |
| Anodized titanium with | Ti 2p3/2 | 458.7 | 67.6 |
| nano-tube structures | Ti 2p1/2 | 464.5 | 32.4 |

TABLE 3
Atomic percentage of selective elements in the outermost
layers of un-anodized and anodized titanium substrates
as examined by X-ray Photoelectron Spectroscopy

| Substrates | C | O | Ti | N | F |
|---|---|---|---|---|---|
| Un-anodized titanium | 43.2 | 41.7 | 8.3 | 2.5 | 2.2 |
| Anodized titanium with | 40.8 | 42.9 | 9.0 | 1.5 | 2.8 |
| nano-tube structures | | | | | |

### 3. Chondrocyte Adhesion

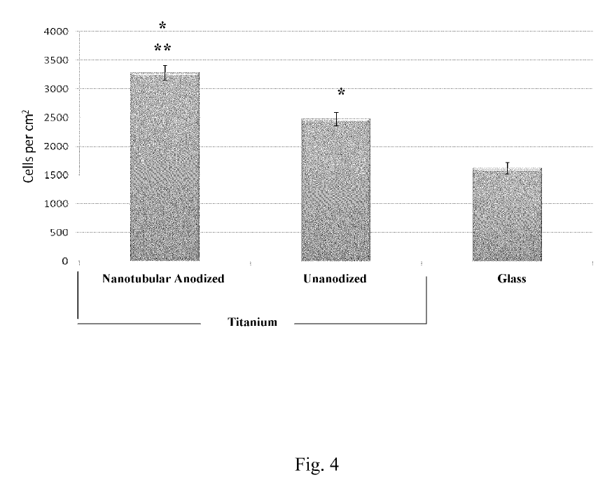

As seen in FIG. 4, greater chondrocyte adhesion on the nanotubular anodized titanium when compared to un-anodized titanium. It is shown that 40% more chondrocytes were counted on anodized titanium compared to un-anodized titanium. Cells were more round on the un-anodized titanium compared to anodized titanium. FIG. 4 shows normalized results as to the surface area provided by AFM characterization studies; thus, they incorporate the greater surface area of the nanotubular anodized titanium and still showed greater chondrocyte adhesion.

The results of performing the inventive process provides evidence as to why chondrocyte adhesion was promoted on nanotubular anodized titanium. Changes in both topography and chemistry after anodization of titanium may influence chondrocyte adhesion. To better understand the role that topography played in this study to promote chondrocyte adhesion, it was necessary to eliminate the influence of chemistry and crystallinity. The disclosed evaluation provides evidence that un-anodized and nanotubular anodized titanium had similar chemistry and crystallinity. It is suggested by the inventors that the nanotubular surface topography resulting from titania anodization process was a major factor that influenced greater chondrocyte adhesion.

In addition, changes in surface topography could also affect surface wettability and surface potential, which are all known to influence chondrocyte responses. It is well understood by one skilled in the art that increased wettability of a surface means that water will spread out more on such a surface. Wettability also is directly related to the surface hydrophilicity (i.e., increased hydrophilicity means greater wettability) and increased surface energy.

Due to the specific titanium surface morphology after anodization, the charge distribution and arrangement on the surface in the culture medium may also be different compared to un-anodized substrates. For example, the more sharp bottoms and edges of the nanotubes on titanium may lead to higher charge densities. Different surface charge densities will lead to different surface electric potential. The Zeta (ξ) potential is the electric potential at an interface between a solid surface and a liquid. In the evaluation disclosed herein, the anodized titanium surface with nanotube structures may have a different Zeta potential compared to the un-anodized titanium with a thinner natural oxide layer. This would also influence initial protein adsorption events responsible for increased chondrocyte adhesion. It has been shown previously that the highest fibronectin adsorption on anodized titanium possessing nanotube structures among the un-anodized and anodized titanium, as well as higher fibronectin adsorption on anodized titanium possessing nano-particulate structures when compared to un-anodized titanium.

By selecting proper anodization conditions, nanotubes can be formed on titanium surfaces with similar chemical composition and crystallinity to the starting un-anodized titanium. The results from using the inventive method shows that enhanced chondrocyte adhesion on nanotubular anodized titanium when compared to un-anodized titanium.

It is understood that the unique nanotube structures provided more surface area and more reactive sites for initial protein interactions that may mediate chondrocyte adhesion. Although the chondrocyte adhesion results were normalized to the increased surface area of nanotubular anodized titanium (See, FIG. 4), changes in protein interactions may promote greater chondrocyte adhesion. It is also contemplated that the unique nanotube structures (inner diameter 70 to 80 nm, a few hundred nm deep) might be sites for preferential adsorption of proteins (vitronectin is 15 nm in length and fibronectin is about 130 nm long to mediate chondrocyte adhesion.

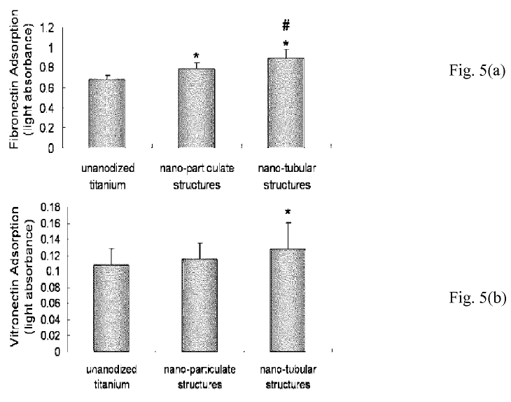

It is a feature of an embodiment of the present invention to provide a surface post-anodization that regulates protein adsorption on the surface. The results shown in FIGS. 5(a) and (b) demonstrate the significant increase of both fibronectin (15%) and vitronectin (18%) adsorption on nano-tubular titanium structures compared to un-anodized titanium samples. Because the cells adhered to the titanium surface via pre-adsorbed proteins, increased fibronectin and vitronectin adsorption on anodized titanium substrates with nano-tubular structures may regulate the observed enhanced cellular functionality.

In another embodiment of the invention, an implant that has undergone the inventive anodization method resulting in the production of surface titanium nanotubes may be an implantable drug delivery system used to deliver pharmaceutical products or other biological agents/materials in vivo. Specifically, the titanium nanotubes may act as carriers and reservoirs to deliver drugs to certain locations of the body over various predetermined time periods.

Anodized titanium with nanotubular structures to deliver drugs, various surface modification techniques were evaluated to determine the release characteristics of various unique and new drug loading methods to be used post-anodization.

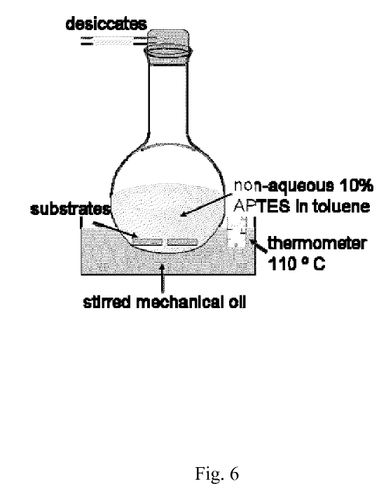

There were three steps of chemical reactions in this evaluation. To introduce hydroxyl groups on anodized titanium, anodized titanium substrates were soaked in a mixture of sulfuric acid and hydrogen peroxide (1:1, Sigma), so called Piranha solution, for 10 minutes (step 1). After that, as shown in FIG. 6, silanization was conducted by immersing samples in 100 ml of a non-aqueous solution of 10% amino-functional organosilane (APTES, Sigma) in toluene (step 2). The reaction was heated by an oil bath at 110° C. for 4 hours. This silanization reaction resulted in the formation of amine groups terminated on anodized titanium surfaces. Finally, some of the samples being evaluated underwent further chemical reactions with acetic anhydrate (Sigma) for 30 minutes with stirring to substitute amine groups with methyl groups (step 3).

After these chemical modification, five different types of titanium substrates were used for the examples discussed below. They included: un-anodized Ti (hereinafter “U”), anodized titanium (hereinafter “A”), anodized titanium terminated with hydroxyl groups (hereinafter “A-OH”), anodized titanium terminated with amine groups (hereinafter “A-NH₂”), and anodized titanium terminated with methyl groups (hereinafter “A-CH₃”).

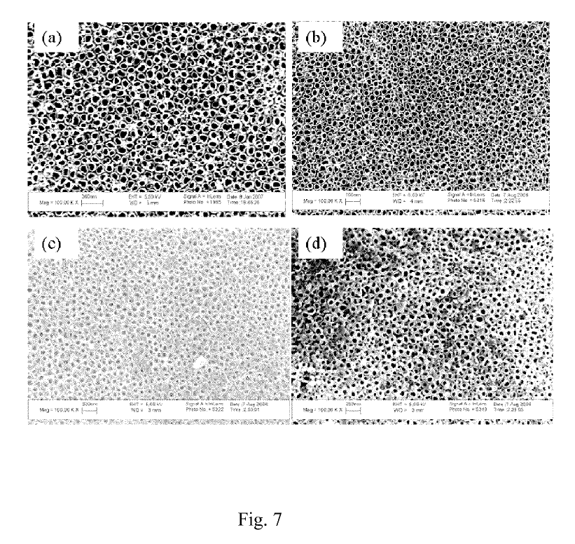

For drug delivery applications, it may be necessary to maintain the nanotubular structures during any surface modifications necessary for loading drugs. As seen in FIGS. 7(a), (b), (c) and (d), under SEM, it was clear that none of the reactions (introducing hydroxyl, amine, and methyl functional groups) significantly changed the nanotubular structures.

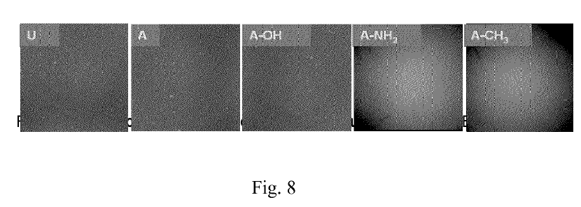

The efficiency of silanization on anodized titanium with nanotubular structures was qualitatively confirmed by the CBQCA reagent kit. FIG. 8 shows fluorescence signals uniformly over the anodized titanium with nanotubular structures where amine groups were introduced. FIG. 8 evidences good efficiency of silanization on the anodized titanium with nanotubular structures. In contrast, none of the un-anodized titanium, unmodified anodized titanium, and anodized titanium terminated with hydroxyl groups showed a fluorescent signal. During the CBQCA assay, the anodized titanium terminated with methyl groups were shown to have good fluorescence intensity, indicating that the efficiency of the step 3 reaction described above may not be high enough to replace all the primary amines with methyl groups.

It is contemplated that numerous drug loading processes may be utilized to fabricate a drug delivery system and medical implant carrier following the performance of the inventive anodization process. Alternative inventive drug delivery systems and corresponding implants that are fabricated after undergoing an innovative drug loading method are described in more detail with references to the following non-limiting examples.

### Example 1

### Drug Physical Adsorption Method

To assess drug loading, anodized titanium substrates of different surface chemistry were immersed into 1 ml of either a P/S solution (containing 6.25 mg penicillin and 10 mg streptomycin per ml) or a P-G sodium salt (6.25 mg penicillin per ml) for a predetermined time (24 hours) under room temperature in a vacuum oven (−20 inch Hg, equaled to −0.67 atmospheric). Samples were then taken out of the oven, rinsed with enough DI water to remove the excessive drug solutions remaining on the surface. These samples were vacuum dried until used. Some of the samples were imaged by a scanning electron microscope (hereinafter “SEM”) to observe the morphology of the drugs adsorbed onto and into titania nanotube structures. The other samples were used for drug release experiments.

### Drug Loading and Release Behavior

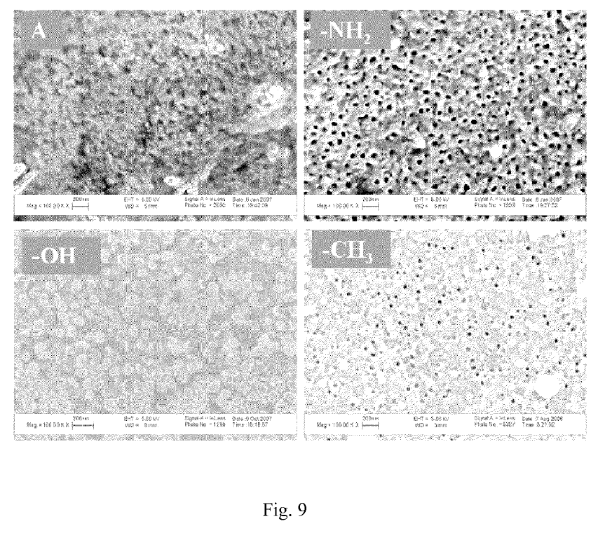

As seen in FIG. 9, after soaking in the P/S or P-G solutions overnight, titanium substrates with different surface chemistry (and, thus, different surface wettability) showed different drug adsorption morphologies under SEM. Generally, there was no uniform coverage over any of the substrates by P/S or P-G except for the A-OH titanium. Unfilled nanotubes can be seen in some areas of A, A-NH₂, and A-CH₃ titanium substrates.

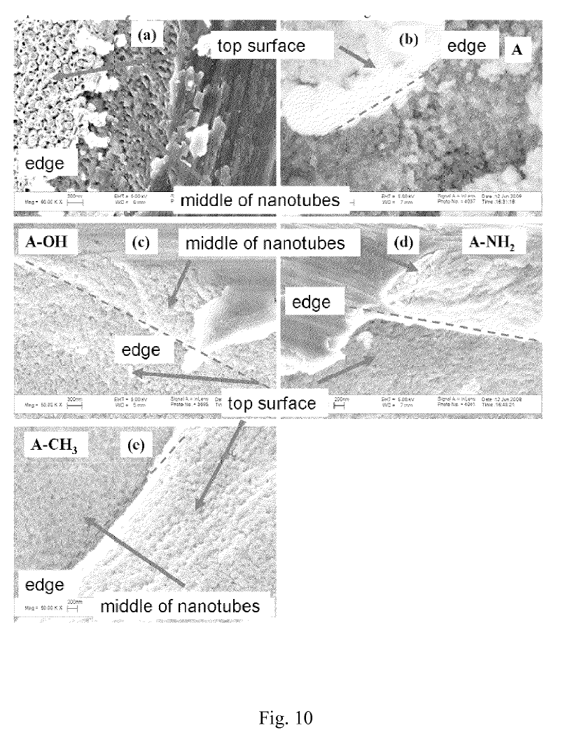

However, the top-view images seen in FIG. 9 do not indicate whether or not the depth of the nanotubes was filled with drug molecules. For this reason, some top portions of the titania nanotubes were mechanically abraded to reveal the deeper portions of the nanotubes. As shown in FIG. 10(a), anodized titania nanotubular structures without loaded drugs were empty. The nanopores on the inclined surface (i.e., the edge area) could be seen since there was nothing loaded into the nanotubes. In other words, the nanopores on the edge area would not be seen if they were filled with drugs. For anodized titanium sample loaded with penicillin seen in FIG. 10(b), some nanopores were seen, which is in agreement with the SEM images of FIG. 9 that not all the nanotubes were filled with drug molecules. However, some of the nanopores were filled and could not be seen. Importantly, for the A-OH titanium samples, no nanopores were seen on both the top and the middle of the nanotubes, indicating their filling with drugs (See, FIG. 10(c)). The A-NH₂ and A-CH₃ titanium samples were similar to the aforementioned anodized samples, with some empty nanopores being seen in FIGS. 10(d) and (e).

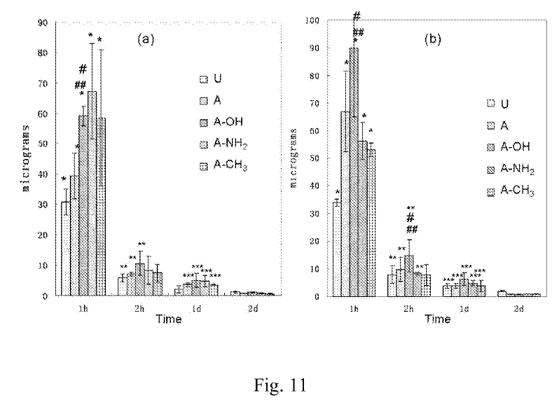

To quantitatively determine how much of a drug was loaded onto/into such titanium substrates, drug release profiles were characterized. As seen in FIG. 11(a) and (b), the release behaviors of the two antibiotics evaluated were very similar to each other. For example, for the A-OH titanium sample it was seen that most of the drug total amount was released within one hour (about 60 μg for P/S and 90 μg for P-G). Then, the released drug amount dropped quickly to around 10 μg for P/S and 15 μg for P-G during the second hour. Finally, the released drug amount decreased to only a few μg or close to zero after two days. The most significant result shown in FIGS. 11(a) and (b) was that anodized titanium terminated with hydroxyl groups outperformed the un-anodized and anodized titanium substrates in terms of drug release amount after 1 hour and 2 hours.

### Example 2

### Drug Electrodeposition Method

Another example method used to load drugs into/onto the various titanium substrates evaluated was cathodic electrodeposition. In this method, titanium substrates (or modified titanium substrates as described above, were used as a cathode in an electrochemical cell similar to that of anodization. A 5% penicillin solution in DI water (P/S or P-G) was used as an electrolyte. 0.9 wt. % NaCl was used as a control electrolyte. The applied voltage was constant at 5 volts or 8 volts according to experimental observations. The deposition time was 5 minutes.

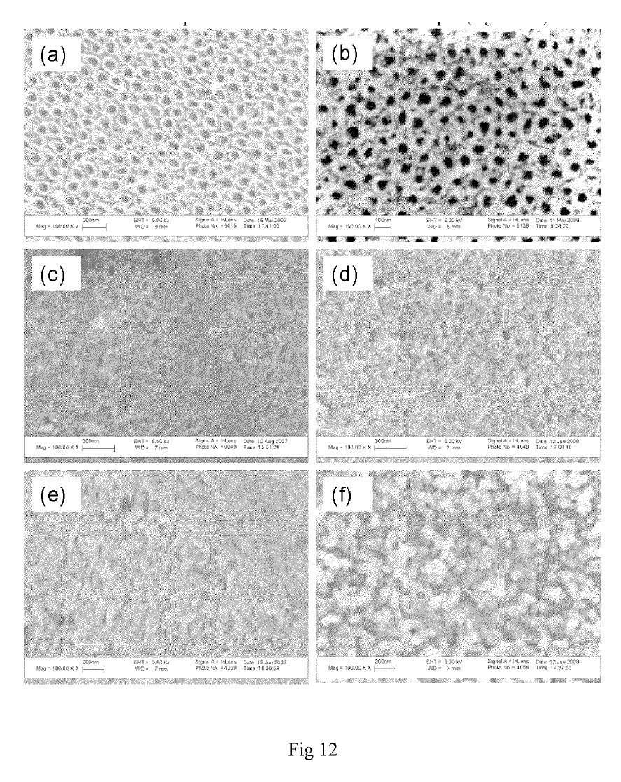

As described above, the anodized titanium with nanotubular structures was used as a cathode in an electrodeposition system to promote drug loading and prolonged drug release from the anodized titanium substrate. Without an applied voltage, it is seen in FIG. 12(a) that close to no drugs were deposited onto the anodized titanium substrates Because the P/S solution contained 0.9% NaCl, an electrolyte containing only NaCl was used to determine the role of sodium salt in this deposition process. It is shown in FIG. 12(b) that some salt crystals would be deposited onto the nanotubes along the edges, but the nanotubes were not capped by such crystals. In comparison, when the electrolyte was P/S and a voltage of 8 volts was applied, the SEM images seen in FIG. 12(c) shows a well covered surface in which the nanotube structures were barely seen. As shown in FIGS. 12(d), (e) and (f), titanium samples A-OH, A-NH₂, and A-CH₃ also had similar results to the anodized titanium samples.

### Drug Loading and Release Behavior

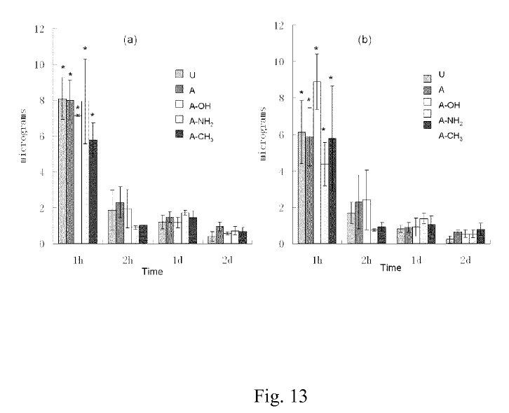

The release of drugs from the electrodeposited titanium substrates was much different from that of the physical adsorption loaded titanium substrates. The total amount of released drugs was less than 15 μg, but the drug released after the first hour was much closer to that released in the first hour than in the titanium substrates that underwent the physical adsorption method. Taking the A-OH titanium sample for example, most of the total amount was released within one hour (about 7 μg for P/S and 9 μg for P-G). Then, the released amount dropped quickly to around 2 μg for P/S and P-G during the second hour. Finally, the released drug amount decreased to less than 1 μg or close to zero after two days. As shown in FIGS. 13(a) and (b), there was no significant difference between A-OH titanium samples and the other anodized titanium samples.

### Example 3

### Drug Co-precipitation with Calcium Phosphate Method

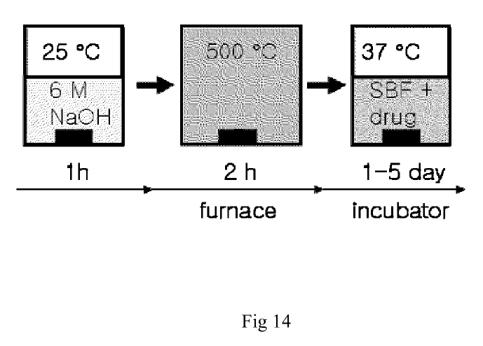

A third example method used to load drug molecules into/onto the various titanium substrates was a co-precipitation method. This method was distinct from Example 1, physical adsorption method and used different post-anodization treatments as denoted in FIG. 14. Specifically, after the cleaning step described above, the anodized titanium samples were soaked in a 6.0 M sodium hydroxide for approximately 1 hour to form sodium titanate on the surface (hereinafter “ASH titanium”). The ASH titanium samples were then removed and placed in a furnace at 500° C. in the air for approximately 2 hours and then were allowed to cool to room temperature in air. Once the ASH titanium samples were prepared they were allowed to soak in 1.5× Simulated Body Fluid (hereinafter “SBF”), containing 11.994 g NaCl, 0.525 g NaHCO₃, 0.336 g KCl, 0.342 g K₂HPO₄.3H₂O, 0.458 g MgCl₂.6H₂O, 0.417 g CaCl₂, 0.107 g Na₂SO₄, and 9.086 g (CH₂OH)₃CNH₂ in 1000 ml dH₂O, pH 7.25) or a mixture of P/S (5 vol. %, 10 vol. %, 20 vol. %) in 1.5×SBF for 3 days. After soaking, they were dried at room temperature overnight and prepared for observation and analysis via the SEM.

### Drug Loading and Release Behavior

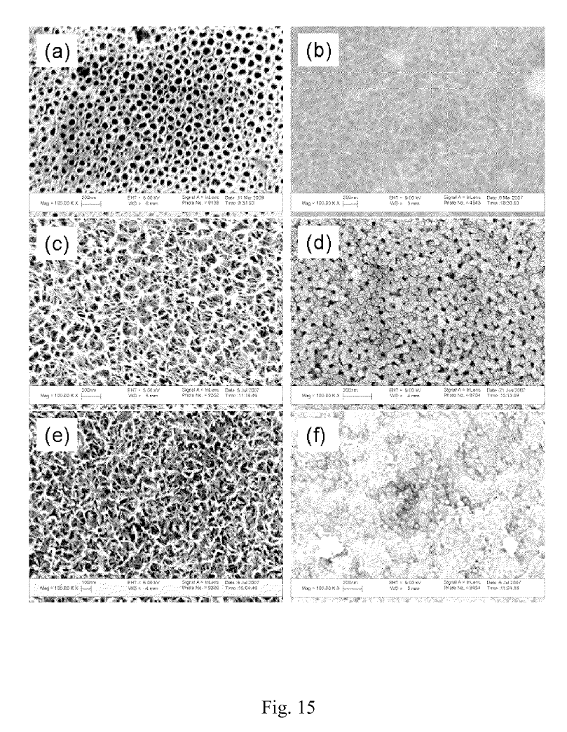

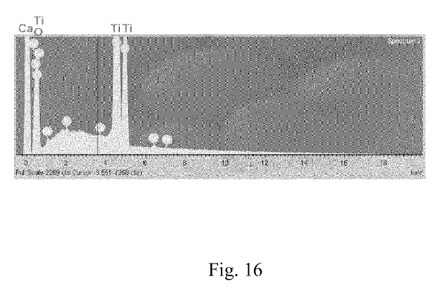

As shown in FIG. 15, the SEM images of the ASH titanium samples that underwent the drug co-precipitation method, showed the appearance of the anodized titanium substrates after they were soaked in sodium hydroxide for 1 hour (See, FIG. 15(b)). It can be also seen that fiber-like crystals formed along the edges of the nanotubes. Energy dispersive spectroscopy (hereinafter “EDS”) results confirmed that these crystals were composed of sodium and titanium; thus, the crystals were considered sodium titanate. After being soaked in 1.5×SBF for 3 days, needle-like minerals were observed on most of the anodized titanium surfaces (See, FIG. 15(c)). These areas were also analyzed by EDS and were found to have calcium and phosphorous (See, FIG. 16). Thus, these minerals were considered to be calcium phosphates. As seen in FIG. 15(d), some areas of the titanium anodized surface exhibited coatings with a different morphology, specifically, a more dense particulate structure. When the anodized titanium substrate were soaked for 3 days in the SBF solution that contained 20% P/S, the substrate exhibited a similar surface morphology to those without P/S (See, FIGS. 15(e) and (f)). Higher P/S concentrations in the SBF solution led to very dense coatings on the anodized titanium surface and, thus, were thought to interrupt the precipitation process of calcium phosphates.

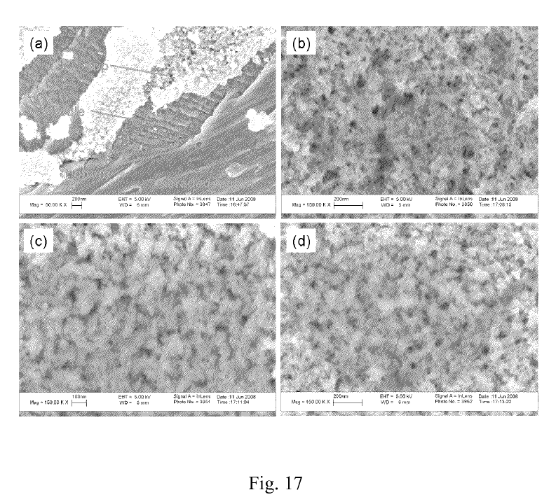

The same abrasion method described above was used for this Example 3 to evaluate the filling of titania nanotubes during co-precipitation. SEM images seen in FIG. 17(a) demonstrated that the co-precipitation of P/S and HA mainly formed on the top of the anodized titania nanotubes as unfilled nanopores were seen in the middle of these titania nanotubes structures.

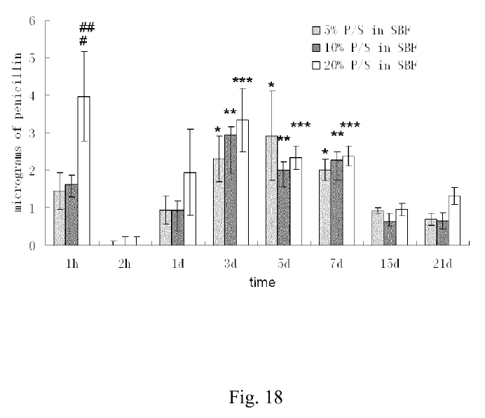

During the drug release evaluation, the anodized titanium substrates were soaked in three concentrations of P/S in SBF solutions at 5%, 10%, and 20% vol. Then, these substrates were used to test the drug release behavior. The results of this evaluation are seen in FIG. 18. Since the drug concentration was as low as 5%, the total release amount was comparable to the electrodeposition method described in Example 2 above and was around 10 to 20 μg. The most obvious difference was that the release of drugs lasted much longer with the Example 3, co-precipitation method than those in the previous two Example (adsorption and electrodeposition) methods. There was significant release within one hour (e.g., 4 μg for the 20% P/S solution), but nearly nothing during the second hour. A major release peak was found in the next week (about 10 μg for the 20% P/S solution) and was not completed until 21 days. From the SEM images seen in FIGS. 17(b)-(d), it is shown that the calcium phosphate minerals remained on the substrates after the soaking step was completed.

It should be noted that the various anodized titanium substrate samples that had undergone the various post-anodization chemical modifications and drug loading methods were also evaluated for bacteria adhesion (anti-bacteria behavior), osteoblast adhesion, surface chemistry changes, contact angles, and surface energy. Performance of these evaluations allowed the inventors to determine the degree of titanium nanotube functionalization and possible in vivo efficacy.

In addition, although not discussed in depth herein, it would be understood by one skilled in the art that by varying the dimensions (e.g. depth, etc.) of the nanostructures, the time period for the drug loaded by the above three loading Examples methodologies may be varied. An additional end use for these three examples may also include the formation of an antimicrobial layer where the anodized nanostructures act to inhibit or destroy the growth of adjacent microbes following implantation of the anodized medical implant.

Yet further end uses of the three Example inventive drug loading methods may include additional functionalizing of the nanostructures with various anti-microbial agents, growth factors, growth agents, or tissue platforms or scaffold to promote tissue ingrowth and apposition. It is contemplated that the inventive anodization process in combination with the disclosed drug loading methods may also be used to promote interaction and increased functionality with a myriad of target tissue and cell types including, but not limited to, cartilage, chondrocytes, ligaments, tendons, entheses, muscle, nerves and other soft-tissue compositions.

Various patent and/or scientific literature references have been referred to throughout the instant specification. The disclosures of these publications in their entireties are hereby incorporated by reference as if completely written herein. In view of the detailed description of the invention, one of ordinary skill in the art will be able to practice the invention as claimed without undue experimentation. Other aspects, advantages, and modifications are within the scope of the following claims as will be apparent to those skilled in the art.

Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A method for producing a plurality of nanostructures on a surface of a medical implant, the method comprising:
presoaking the implant in a solution;
providing an anodization electrolyte solution;
providing a cathode;
submerging the cathode and medical implant in the electrolyte solution;
applying a voltage for a set time period between the medical implant and the cathode to generate a plurality of nanostructures on the surface of the medical implant; and
removing the medical implant from the electrolyte solution and rinsing the surface of the medical implant.

2. The method of claim 1, wherein the presoaking solution comprises deionized water, hydrofluoric acid and nitric acid.

3. The method of claim 1, wherein the plurality of nanostructures comprises nanotubes.

4. The method of claim 1, wherein the medical implant comprises titanium or a titanium alloy.

5. The method of claim 1, wherein the anodization electrolyte solution comprises a fluorine based acidic solution.

6. The method of claim 5, wherein the fluorine based acidic solution comprises hydrofluoric acid and nitric acid.

7. The method of claim 1, wherein the voltage applied between the medical implant and the cathode is constant for the set time period with a magnitude of between 1 volt and 25 volts.

8. A method for fabricating a medical implant with increased chondrocyte functionality, the method comprising:
obtaining a medical implant, the medical implant being fabricated from at least one of a metallic material, a polymer, a ceramic and a composite; and
treating a surface of the medical implant to modify the surface topography resulting in increased chondrocyte functionality.

9. The method of claim 8, wherein the medical implant is fabricated from titanium or titanium alloy.

10. The method of claim 8, wherein the treating the surface of the medical implant comprises anodizing the surface to create a plurality of nanostructures, the nanostructures being configured to increase chondrocyte functionality.

11. The method of claim 10, wherein the plurality of nanostructures comprise a plurality of titanium oxide nanotubes.

12. The method of claim 11, wherein the inner diameters of the titanium oxide nanotubes on the surface of the medical implant are between 40 and 90 nm.

13. The method of claim 11, wherein the depth of the titanium oxide nanotubes on the surface of the medical implant is between 100 and 500 nm.

14. The method of claim 10, wherein anodizing the surface of the medical implant increases the surface wettability, the increased wettablity causing increased chondrocyte adhesion to the surface of the medical implant.

15. A method for fabricating a drug delivery system for use in a living body, the method comprising:
obtaining a medical implant, the medical implant being fabricated from at least one of a metallic material, a polymer, a ceramic and a composite; and
treating a surface of the medical implant to modify the surface topography resulting in increased surface roughness, thereby fabricating a system by which a biological material or a pharmaceutical product can be retained and delivered to a part of a living body.

16. The method of claim 15, wherein the medical implant is fabricated from titanium or titanium alloy.

17. The method of claim 15, wherein the treating the surface of the medical implant comprises anodizing the surface to create a plurality of nanostructures, the nanostructures being configured to retain a biological material or a pharmaceutical product for delivery to a part of a living body.

18. The method of claim 17, wherein anodizing the surface to create a plurality nanostructures comprises:
presoaking the medical implant in an acidic solution;
providing an anodization electrolyte solution;
providing a cathode;
submerging the cathode and medical implant in the electrolyte solution;
applying a voltage for a set time period between the medical implant and the cathode to generate a plurality of nanostructures on the surface of the medical implant; and
removing the medical implant from the electrolyte solution and rinsing the surface of the medical implant.

19. The method of claim 17, wherein the plurality of nanostructures comprise a plurality of titanium oxide nanotubes.

20. The method of claim 17, wherein the biological material or pharmaceutical product is at least one of an anti-microbial agent, protein, growth factor, bone morphogenic protein, ceramic, growth agent, tissue platform, stem cell, tissue scaffold element, anti-inflammatory agent, antibiotic agent, antiviral agent, antigen, allograft, and enzyme.

21. The method of claim 15, further comprising loading the medical implant with the biological material or pharmaceutical product.

22. The method of claim 21, wherein the loading the medical implant comprises performing at least one of a physical adsorption method, an electrodeposition method and a co-precipitation with ceramic method.

23. A device for delivering a drug or biologic agent within a living being comprising, a medical implant with a surface, wherein integrally attached to the surface are a plurality of nanostructures, the nanostructures being configured to retain or adsorb the drug or biologic agent.

24. The device of claim 23, wherein the plurality of nanostructures are a plurality of nanotubes.

25. The device of claim 24, wherein the inner diameter of each the plurality of nanotubes is between 40 and 90 nm.

26. The device of claim 24, wherein the depth of the plurality of nanotubes is between 100 and 500 nm.

27. The device of claim 23, wherein the medical implant comprises titanium or titanium alloy.

28. The device of claim 23, wherein the plurality of nanostructures retain or adsorb the drug or biologic agent after undergoing at least one of a physical adsorption method, an electrodeposition method and a co-precipitation with ceramic method.

29. A medical implant having a surface configured for regulating protein adsorption, the surface comprising a plurality of nanostructures, the nanostructures being formed and integrally attached to the surface following the implant undergoing a surface treatment process before implantation into the body.

30. The medical implant of claim 29, wherein the medical implant comprises titanium or titanium alloy.

31. The medical implant of claim 29, wherein the plurality of nanostructures are a plurality of nanotubes.

32. The medical implant of claim 29, wherein the surface treatment process comprises:
presoaking the medical implant in an acidic solution;
providing an anodization electrolyte solution;
providing a cathode;
submerging the cathode and medical implant in the electrolyte solution;
applying a voltage for a set time period between the medical implant and the cathode to generate a plurality of nanostructures on the surface of the medical implant; and
removing the medical implant from the electrolyte solution and rinsing the surface of the medical implant.

33. The medical implant of claim 31, wherein the inner diameter for each of the plurality of nanotubes is between 40 and 90 nm.

34. The medical implant of claim 31, wherein the depth of the plurality of nanotubes on the surface of the medical implant is between 100 and 500 nm.

35. The medical implant of claim 29, wherein the surface treatment process increases at least one of the surface wettability and the surface energy, at least one of the increased wettability and the surface energy causes an increase in protein adsorption to the surface of the medical implant.

36. The medical implant of claim 35, wherein the rate of protein adsorption is regulated by at least one of the size of each of the plurality of nanotubes and the depth of the plurality of nanotubes integrally attached to the surface of the medical implant.

37. The medical implant of claim 35, wherein the rate of fibronectin or vitronectin adsorption is regulated by at least one of the size of each of the plurality of nanotubes and the depth of the plurality of nanotubes integrally attached to the surface of the medical implant.
